(19) United States
(12) Patent Application Publication
(10) Pub. No.: US 2024/0215866 A1
(43) Pub. Date: Jul. 4, 2024

(54) HEAD-MOUNTED DEVICES FOR POSTURAL ALIGNMENT CORRECTION

(71) Applicant: Microsoft Technology Licensing, LLC, Redmond, WA (US)

(72) Inventors: Rayna DEMASTER-SMITH, Kirkland, WA (US); Mehdi ZIAT, Seattle, WA (US)

(73) Assignee: Microsoft Technology Licensing, LLC, Redmond, WA (US)

(21) Appl. No.: 18/147,307

(22) Filed: Dec. 28, 2022

(51) Int. Cl. A61B 5/11; A61B 5/00; G06V 10/82; G06V 40/10

(52) U.S. Cl. CPC A61B 5/1116; A61B 5/1121; A61B 5/6803; A61B 5/742; G06V 10/82; G06V 40/10

(57) ABSTRACT

Examples of head-mounted devices for postural alignment correction are provided. In one aspect, a head-mounted device is implemented to include a display, a plurality of sensors including an inertial measurement unit, and a controller. The controller includes instructions executable to control the head-mounted device to receive inertial measurement data from the inertial measurement unit, to input the received inertial measurements into a machine learning model, and to receive an estimated body posture from the neural machine learning model. In this aspect, additionally or alternatively, the machine learning model is an artificial neural network that has been trained to estimate the body posture by calculating a craniovertebral angle, where the craniovertebral angle is defined as an angle between a first line crossing a tragus point and a cervical vertebra point and a second line crossing the cervical vertebra point.

### BACKGROUND

A person's natural head posture can affect his or her health. Poor head posture has been shown to be correlated with various types of physical ailments, including respiratory problems, headaches, and pain. In general, poor head posture is characterized as a forward head posture that can vary in severity. A more pronounced forward position puts increasing amounts of weight pressure on the spine. Forward head posture is caused by activities that result in a person leaning his or her head forward for prolonged periods of time. For example, in the current technological age, forward head posture is often associated with the use of electronic devices, such as cell phones and computers. Use of such devices professionally or leisurely for prolonged periods of time can lead to chronic forward head posture. Treatment for problems caused by chronic forward head posture includes physical therapy and, in severe cases, surgery. For the physical therapy route, it is recommended to perform certain exercises and stretches on a routine basis to help loosen stiff muscles and joints of the affected areas.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

Examples of head-mounted devices for postural alignment correction are provided. In one example, a head-mounted device is implemented to include a display, a plurality of sensors including an inertial measurement unit, and a controller. The controller includes instructions executable to control the head-mounted device to receive inertial measurement data from the inertial measurement unit, to input the received inertial measurements into a machine learning model, and to receive an estimated body posture from the machine learning model. In this aspect, additionally or alternatively, the machine learning model is an artificial neural network that has been trained to estimate the body posture by calculating a craniovertebral angle, where the craniovertebral angle is defined as an angle between a first line crossing a tragus point and a cervical vertebra point and a second line crossing the cervical vertebra point.

### BRIEF DESCRIPTION OF THE DRAWINGS

FIG. 1 shows a schematic view of an example computing system for estimating body posture using a head-mounted device.

FIG. 2 schematically shows an example machine learning model for estimating body posture.

FIG. 3 schematically shows an example methodology for estimating body posture by calculating a craniovertebral angle.

FIGS. 4A and 4B schematically show an example methodology for estimating body posture using downward-facing cameras.

FIG. 5 shows an example head-mounted device for estimating body posture.

FIG. 6 shows a flow diagram of an example method for estimating body posture.

FIG. 7 shows a block diagram of an example computing system that can enact one or more of the methods and processes described in FIGS. 1-6.

### DETAILED DESCRIPTION

Forward head posture occurs when a person is leaning their head forward, out of neutral alignment with his or her spine. A person's natural head posture may gravitate towards a forward head posture when he or she regularly partake in activities that result in leaning his or her head forward for prolonged periods of time. Preventative measures include maintaining proper posture, i.e.—keeping the head aligned vertically with the spine, during these activities. However, focusing on maintaining proper posture can be difficult for certain activities. For example, in an office setting, a person may sit at a desk for long periods of time. Focus on keeping proper posture throughout the day may be difficult to maintain, as the body tends to return to a position that requires less physical effort to maintain.

Various posture correction devices are available commercially for use in preventing and treating poor posture. These devices include brace-like structures and smart devices for assisting users to maintain proper posture during the activities described above. Braces help the user to retain a proper posture by providing resistance against a poor posture position. Wearable smart devices, often worn on the user's back, detects a current poor posture position and alerts the user accordingly to take proactive measures to correct his or her posture. However, both categories of posture corrective devices involve uncomfortable and restricting wearables that users often find unacceptable.

In view of the observations above, examples of head-mounted devices for detecting body posture, such as head posture, hip posture, spinal posture, etc., and related implementations are provided in the present disclosure. As opposed to clinical settings where objective assessment of poor body posture, such as forward head posture, includes bulky equipment and precise calibration steps, estimating body posture using head-mounted devices can be conveniently implemented as everyday wearables. For example, head-mounted devices can be implemented as head-mounted displays in various forms, including smart eyeglasses, goggles, etc. Such devices can be adopted as everyday wearables that the user is likely to be wearing during activities in which the user's head is leaning forward for a prolonged period of time.

Example methods for estimating body posture using head-mounted devices include the use of various sensors attached on such devices. Example sensors include gyroscopes, accelerometers, magnetometers, inertial measurement units (IMUs), cameras, capacitive/inductive antennas, etc. Example methods for estimating head posture alternatively or additionally can include the use of cameras onboard the head-mounted device. The cameras can be oriented and positioned in various ways. For example, the cameras can be implemented as forward-facing cameras or downward-facing cameras. As the user's head vertically tilts, the cameras are further pivoted such that more of the user's body is in view of the camera. Such information can be used to infer body posture. For example, the information can be used to estimate a craniovertebral angle to infer forward head posture. Various ranges of craniovertebral angles can be correlated with forward head posture as well as several other postural misalignments, including pelvic tilt, lumbar lordosis, and sacral slope.

The methodologies described above can be used in combination with deep learning techniques. Data from the sensors can be utilized with a machine learning model to estimate and classify different body postures, including forward head posture. In some implementations, an artificial neural network is implemented to predict body posture using sensor data received from the sensors. Other machine learning models can also be utilized. Examples include clustering algorithms, decision trees, regressions, etc. The body posture estimation process can be performed onboard the head-mounted device or remotely. For example, the body posture estimation process can be performed on a computing device onboard the head-mounted device. In other implementations, the body posture estimation process is performed on a remote computing device, and the predicted body posture information is sent to the head-mounted device.

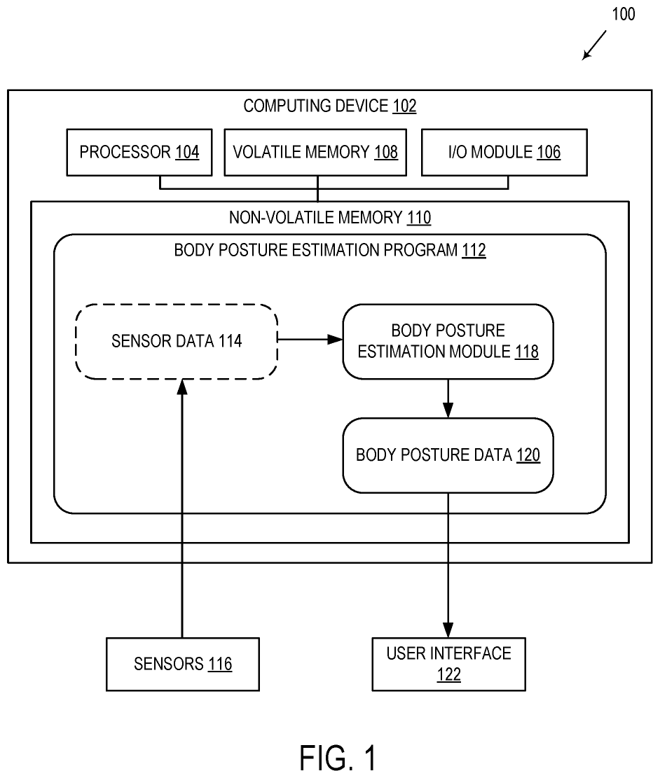

FIG. 1 shows a schematic view of an example computing system 100 for estimating body posture using a head-mounted device. As shown, the computing system 100 includes a computing device 102 that further includes a processor 104 (e.g., central processing units, or “CPUs”), an input/output (I/O) module 106, volatile memory 108, and non-volatile memory 110. The different components are operatively coupled to one another. The non-volatile memory 110 stores a body posture estimation program 112, which contains instructions for the various software modules described herein for execution by the processor 104.

Upon execution by the processor 104, the instructions stored in the body posture estimation program 112 cause the processor 104 to initialize the body posture estimation process, which includes retrieving sensor data 114 from a plurality of sensors 116. Different types of sensor data 114 and associated sensors 116 may be utilized. Example sensor data includes inertial measurement data and image data. Inertial measurement data can be received from an accelerometer, a gyroscope, and/or a magnetometer of an inertial measurement unit. Image data can be received from one or more cameras. Such cameras may be configured to sense intensity (e.g., color, grayscale, infrared, or other wavelength bands), and/or depth. Depth sensing can be performed by a stereo camera arrangement of one or more depth cameras, such as a time-of-flight depth camera. The body posture estimation process can be implemented differently depending on the type of sensor data 114 available. In some implementations, a head tracking system is implemented using cameras and IMUs, such as those described above, to provide head pose data to the body posture estimation program 112. For example, a head tracking system can be implemented using a pair of forward-facing cameras, such as RGB or depth cameras, and at least one IMU to track the user's head pose.

The body posture estimation program 112 includes a body posture estimation module 118 that receives the sensor data 114 as input. In some implementations, the body posture estimation module 118 comprises a machine learning model that utilizes deep learning techniques to estimate body posture based on one or more types of sensor data. Body posture, such as head posture, hip posture, spinal posture, etc., can be estimated using various methods. For example, the body posture estimation program 112 can determine a craniovertebral angle to infer head posture, as some craniovertebral angles can be correlated with more severe forward head posture. In some implementations, the body posture estimation program 112 uses head pose data received from a head tracking system to infer head posture. Alternatively or additionally, the body posture estimation program 112 can use data from at least one camera to estimate head tilt based on image data. Example types of cameras include forward-facing cameras and downward-facing cameras. Such cameras can also be configured to sense hand gestures. Head tilt can be used to infer a forward head posture. One or both of these methods can be used to model an artificial neural network for predicting body posture.

The body posture estimation module 118 outputs body posture data 120 to a user interface 122. The body posture data 120 describes a predicted body posture of the user. For example, the body posture data 120 can include a value indicating a severity of the user's forward head posture. In other implementations, the body posture data 120 indicates whether the user's body posture is in a poor posture position based on a predetermined threshold. For example, a forward head posture position can be determined based on a forward head position threshold. In some implementations, the threshold is determined based on a calibration performed for a particular user. As different users have different anatomies, calibration for a given user can be performed to determine the sensor data associated with a poor posture versus a proper posture. For example, in implementations utilizing sensor data from IMUs, the calibration process can include instructing the user to hold a specified posture (e.g., that corresponds to a proper head posture) and recording the sensor data from the IMUs while the user is in the instructed posture. The recorded sensor data can be set as a baseline for proper posture, and future detected sensor data indicating deviation from such a position can be used to infer a poor posture, such as a forward head posture.

The body posture data 120 can be formatted and presented to the user. For example, on head-mounted devices, the body posture information can be presented to the user via a graphical user interface on a display. The presented information can inform the user of the user's current body posture. In some implementations, the information is presented when the predicted body posture is classified as a poor posture, which can be determined, for example, based on a forward head position threshold. In further implementations, information about corrective measures is also presented to the user. Corrective measures describing stretches and other exercises can be provided in the form of text, images, and/or videos. Certain activities can affect the accuracy of the body posture information. For example, body posture predictions can be inaccurate when the user is in a reclining position. As such, the estimation of body posture can be modified accordingly or halted when a reclining position is detected. Reclining positions can be detected using various sensors on the head-mounted device, including IMUs and cameras.

In implementations where the body posture estimation module 116 includes a machine learning model, different learning techniques can be applied depending on the application. For example, an artificial neural network can be implemented with supervised learning techniques to predict body posture for a given user. In further examples, the artificial neural network is initially a neural network that has been trained using training data. The training data can be associated with an average population or a target demographic, such as age, gender, country of origin, etc. Supervised learning techniques can include the user providing feedback indicating the accuracy of a predicted body posture. The feedback serves as a target output. The artificial neural network then computes a loss value using the target output and the predicted body posture based on a loss function, such as an L1 or L2 loss function. The computed loss can then be used to adjust parameters and/or weights of the artificial neural network, for example using back propagation. The process continues iteratively, and the artificial neural network is continually trained as the body posture estimation module 118 continues to operate. Feedback from the user can be provided in various ways. For example, feedback from the user can be provided to the computing device 102 via the I/O module 106 in various forms. Examples input methods include hand gestures, head tracking, gaze detection, etc.

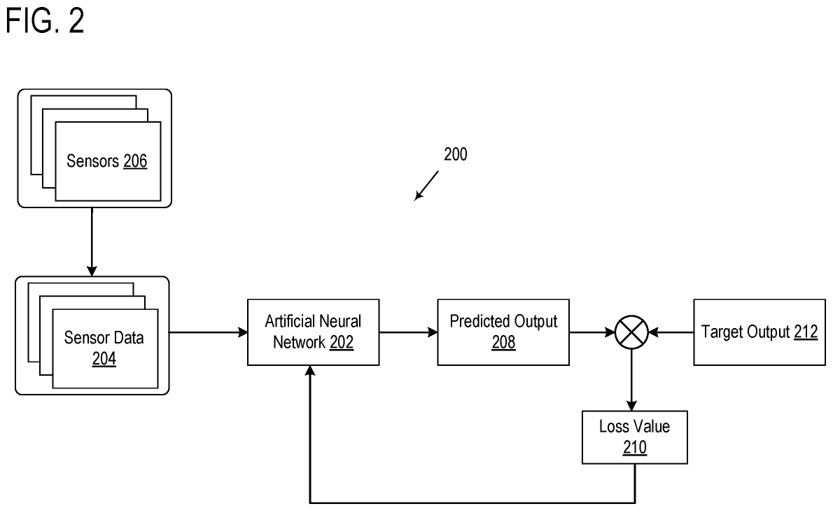

Referring to FIG. 2, an example machine learning model 200 for estimating body posture is schematically illustrated. The machine learning model 200 is implemented using an artificial neural network 202 with supervised learning techniques. The body posture estimation process starts with a plurality of sensor data 204 as input to the artificial neural network 202. The plurality of sensor data 204 can include data from one or multiple sensors 206. Similar or different types of sensors can be utilized. For example, the sensor data 204 can include inertial measurement data from at least one IMU sensor and/or image data from at least one camera. The artificial neural network 202 is designed to output a body posture as a predicted output 208 using the sensor data 204 as input. The artificial neural network 202 can be pre-trained with training data that includes examples of inputs and associated target outputs. The training data can be based on an average of the population or a target demographic, including age, gender, country of origin, etc. For head-mounted devices, the artificial neural network 202 can be implemented such that it is further trained during operation by a user, allowing for increased accuracy in the body posture estimation process for said user. In such cases, the user can provide feedback regarding the accuracy of the predicted body posture.

Training of the artificial neural network 202, including both before and during real-time operation of the artificial neural network 202, can be performed by computing a loss value 210. The loss value 210 is computed based on a measured difference between the predicted output 208 and a target/correct output 212. During the pre-training phase, the target output 212 is provided in the training data. During training of the artificial neural network 202 while implemented on a head-mounted device, the target output 212 is provided in the form of feedback from the user during real-time operation. The loss value 210, which can also be referred to as an error value, is used to adjust the parameters and/or weights of the artificial neural network 202. The process repeats, and the artificial neural network 202 is further trained at each iteration with an end goal of a neural network having more accurate predictions with the given input space.

Methodologies for body posture estimation, whether implemented in an artificial neural network such as the one described in FIG. 2 or in a determinative system, can include one or a combination of processes. One example includes inferring body posture using craniovertebral angles. Relatively smaller craniovertebral angles can be correlated with relatively more severe forward head posture. A craniovertebral angle can be defined in several ways. One definition is an angle formed from a horizontal line crossing a cervical vertebra and a line joining a tragus of the ear to the cervical vertebra. Other methods use a specific cervical vertebra, such as the C7 vertebra, or other anatomical points near the base of the user's neck.

In some examples, estimation of a craniovertebral angle includes the estimation of various anatomical points for a given user. Anatomical points can include estimated points in three-dimensional space associated with a user's anatomy. The anatomical points can be estimated and tracked along with the user's movements using various sensors. Example sensors for estimating and/or tracking such anatomical points include one or more accelerometers, gyroscopes, and/or magnetometers. In some implementations, at least one IMU that includes an accelerometer, a gyroscope, and/or a magnetometer is used to track the anatomical points. As described above, the IMU(s) can be mounted on a head-worn smart device, such as smart glasses. The IMU(s) may be calibrated for a given user, and sensor data from the IMU(s) can be used to infer head movement and posture. For example, an estimate of the craniovertebral angle of the user can be obtained using the determination of the gravity vector by an accelerometer in the IMU. Orientation measurements of the head position can be determined using the rate output of the gyroscope. Further refinements can be made using the output of the magnetometer by measuring the orientation of the head-worn device with respect to the Earth's magnetic field.

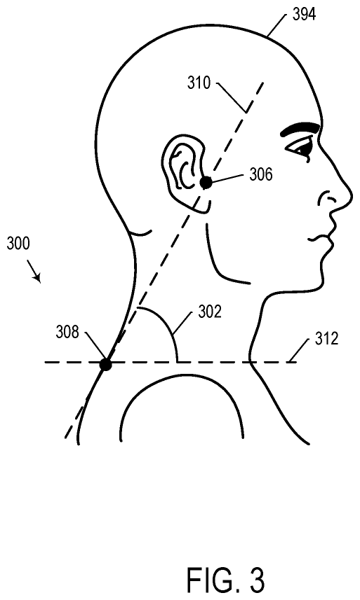

Referring to FIG. 3, an example methodology 300 for calculating a craniovertebral angle 302 is schematically illustrated. The methodology 300 includes first determining various anatomical points on a user 304. In the depicted methodology, the craniovertebral angle 302 is calculated using a tragus point 306 and a C7 vertebra point 308. The points can be determined in various ways. For example, the C7 vertebra point 308 in the example of FIG. 3 is determined based on the location of the spinous process of the C7 vertebra. The craniovertebral angle 302 is formed from a first line 310 crossing the tragus point 306 and the C7 vertebra point 308 and a second line 312 crossing the C7 vertebra point 308. In many implementations, the second line 312 is a horizontal line extending from the C7 vertebra point 308.

The anatomical points can be predetermined through a calibration process, and various sensors can be used to track the user's head movements. Movement of the anatomical points can be inferred from the user's head movements, and body posture information is estimated accordingly. For example, one or more calibrated IMUs can be used to provide measurements describing the force, acceleration, and/or angular position experienced by the IMUs, which are associated with a known position relative to the user's frame of reference. This information can be used to infer changes in the positions of the determined anatomical points.

Another methodology of estimating body posture includes the use of image data from cameras located on head-mounted devices. Different types of cameras can be utilized. Example types of cameras include downward-facing cameras for hand-tracking, outward-facing cameras for environmental tracking, etc. The specific methodology utilized can determine the type of camera implemented. For example, eye-tracking cameras can be utilized to estimate body posture based on gaze detection. Another example includes the use of a downward-facing camera to estimate body posture based on image data indicating the portion of the user's body that is in view of said camera. Generally, a more severe head tilt will result in the downward-facing camera, which is affixed to a head-mounted device, being pivoted further such that more of the user's body will be in view of said camera. Such information can be used to estimate body posture.

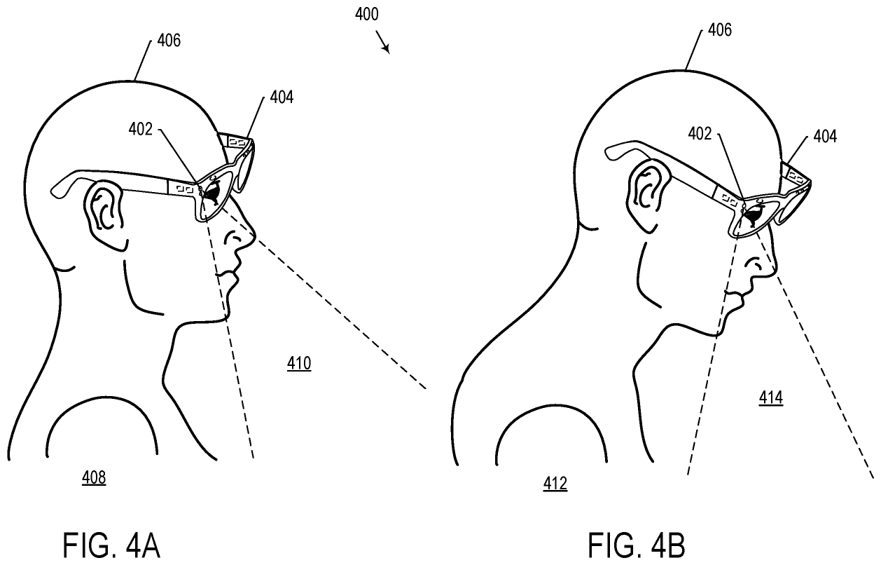

Referring to FIGS. 4A and 4B, an example methodology 400 for estimating body posture using downward-facing cameras 402 is schematically illustrated. As shown, the downward-facing cameras 402 are affixed onto a head-mounted device 404 worn by a user 406. Depending on the user's vertical head tilt, the downward-facing cameras 402 is pivoted accordingly. Generally, a more severe forward head position results in a more pronounced vertical head tilt. As such, the orientation of the downward-facing cameras 402 can be used to infer body posture, such as head posture, for example. FIGS. 4A and 4B respectively illustrate two example head tilt positions and the associated views of the downward-facing cameras 402. The first illustrated head tilt position 408 depicts the user 406 in a proper head posture with little to no forward head lean. In such a position, the downward-facing cameras 402 captures a first view 410. The second illustrated head tilt position 412 depicts the user 406 in a forward head posture. The downward-facing cameras 402 is pivoted further downward relative to its position in the first head tilt position 408. In the second illustrated head tilt position 410, the downward-facing cameras 402 captures a second view 414 different from the first view 410.

Depending on the user 406 and the orientation of the head-mounted device 404, the first view 410 can capture a portion of the user's body. Since the downward-facing cameras 402 and the head-mounted device 404 are in a fixed position relative to the user's head, a forward head position such as the one depicted in the second illustrated head tilt position 412 results in the downward-facing cameras 402 having a view that captures a larger portion of the user's body compared to first view 410. As such, changes to the portion of the user's body that is in view of the downward-facing cameras 402 can be used to infer body posture. In some implementations, a calibration process is performed to inform the system of the type of image data that is associated with a proper body posture. For example, the calibration process can include instructing the user to be in a proper body posture, and image data from the downward-facing cameras 402 is recorded. The system associates this image data with a proper body posture, and changes in future image data where a larger portion of the user's body is in view of the downward-facing cameras 402 will inform the system that the user's head is leaning forward. As can readily be appreciated, different types of cameras, including forward-facing cameras, can also be implemented to estimate body posture.

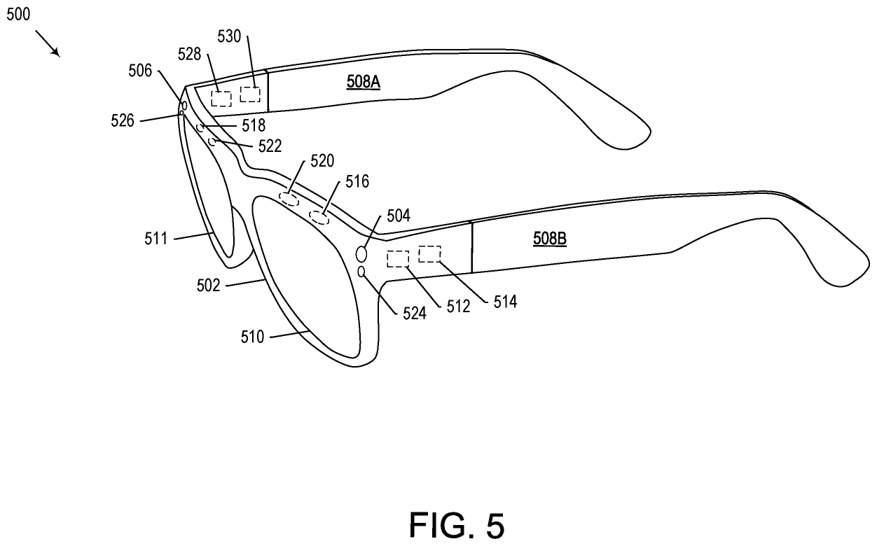

FIG. 5 schematically illustrates an example head-mounted device 500 for estimating head posture. The example head-mounted device 500 includes a frame 502, a first camera 504, a second camera 506, a display, and temple pieces 508. In this example, the display includes a first display 510 and a second display 511 supported by the frame 502, wherein each of the first display 510 and the second display 511 takes the form of a waveguide configured to deliver a projected image to a respective eye of a user. The first camera 504 and the second camera 506 in this example are located respectively at left and right sides of the frame 502, wherein each of the first camera and the second camera is located on the frame adjacent to an outer edge of the frame. Different types of cameras, such as forward-facing and downward-facing cameras, can be implemented.

The head-mounted device 500 may further include other sensors that include aligned left and right components. For example, head-mounted device 500 includes an eye-tracking system. The eye-tracking system includes a first eye-tracking camera 516 and a second eye-tracking camera 518. The head-mounted device 500 further includes a face-tracking system that includes a first face-tracking camera 520 and a second face-tracking camera 522, and a hand-tracking system that includes a first hand-tracking camera 524 and a second hand-tracking camera 526. In some implementations, the head-mounted device 500 implements a pair of cameras, such as forward-facing or downward-facing cameras, to capture image data for both body posture estimation and hand-tracking. Such configurations enable head-mounted devices where the user can provide hand gestures as input without having to hold their hands in view of front-facing cameras.

Data signals from the eye-tracking system, the face-tracking system, and/or the hand-tracking system may be used, in addition to data signals from the first camera 504 and the second camera 506, to provide sensor data for estimating body posture. Additionally, the data signals can be used to detect user inputs and to help render a stereo image in various examples. User inputs can be detected to provide feedback for a predicted body posture, for example. The various camera systems can also be used to provide information for determining whether the user is in an upright or reclining position.

The head-mounted device 500 further includes a first display module 512 positioned adjacent to the first camera 504 for displaying a first image of the stereo image and a second display module 528 positioned adjacent to the second camera 506 for displaying a second image of the stereo image. Each display module may include any suitable display technology, such as a scanned beam projector, a microLED (light emitting diode) panel, a microOLED (organic light emitting diode) panel, or a LCoS (liquid crystal on silicon) panel, as examples. Further, various optics, such as the above-mentioned waveguides, one or more lenses, prisms, and/or other optical elements may be used to deliver displayed images to a user's eyes.

In addition to cameras, the head-mounted device 500 can further include other types of sensors. For example, the head-mounted device 500 includes an inertial measurement unit system (IMU) that includes a first IMU 514 positioned adjacent to the first display module 512 and a second IMU 530 positioned adjacent to the second display module 528. As described in the sections above, the IMUs 514, 530 can be used to provide IMU data for predicting body posture. First camera 504, first display module 512, and first IMU 514 may be closely mechanically coupled to help prevent changes in alignment from occurring between the first camera 504, the first display module 512, and the first IMU 514. Second camera 506, second display module 528, and second IMU 530 may be similarly closely mechanically coupled. IMU data can be used to adjust a displayed image based upon head motion. IMUs 514 and 530 also can be calibrated with a bending moment applied to the head-mounted device 500.

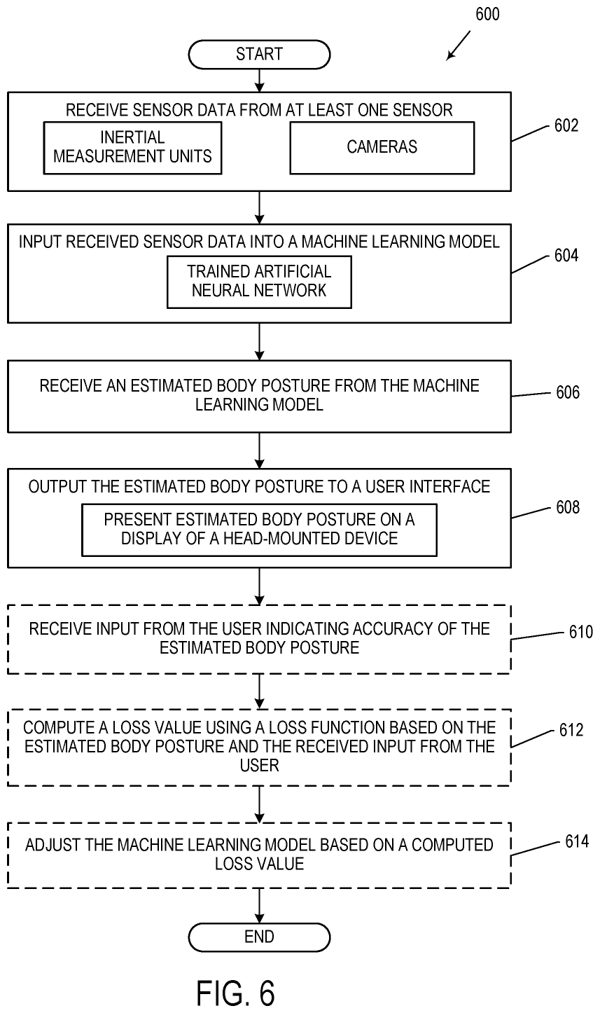

FIG. 6 schematically illustrates a flow diagram of an example method 600 for estimating body posture. The method 600 includes, at step 602, receiving sensor data from at least one sensor. Different types of sensor data can be received from different associated sensors. For example, inertial measurement data and image data can be received from an inertial measurement unit and a camera, respectively. The inertial measurement unit can include different measurement devices, such as an accelerometer, a gyroscope, a magnetometer, etc. Different types of cameras, including forward-facing cameras, downward-facing cameras, eye-tracking cameras, and hand-tracking cameras, can be utilized. Multiples of the same or a combination of many different sensors can be utilized. In some implementations, the method 600 includes receiving inertial measurement data from at least two IMUs. Alternatively or additionally, the method 600 includes receiving image data from one or more downward-facing cameras. In some implementations, the method 600 is performed on a computing device located onboard a head-mounted display. In such cases, the sensors from which data is received are also located on the head-mounted display.

At step 604, the method 600 includes inputting the received sensor data into a machine learning model. In some implementations, received inertial measurements are inputted into an artificial neural network. The artificial neural network may be located on a computing device located on a head-mounted display on which the method 600 is performed. In other implementations, the artificial neural network is located on a remote server. The artificial neural network is designed to predict body posture, such as head posture, hip posture, spinal posture, etc. Methodologies for predicting body posture can include one or a combination of various processes. An example methodology includes calculating a craniovertebral angle to infer body posture. Smaller craniovertebral angles are correlated to more severe forward head posture. Another example methodology includes using image data from a camera, such as a forward-facing camera and/or a downward-facing camera, to infer body posture based on how much of the user's body is in view of the camera. The artificial neural network can be pretrained. The artificial neural network can be trained using training data containing sets of sensor data inputs and target body posture outputs. The training data can represent an average sample of the population. In some implementations, the training data represents a target demographic, such as age, gender, country of origin, etc.

At step 606, the method 600 includes receiving an estimated body posture from the machine learning model. The estimated body posture can include information describing the head position of the user. For example, the estimated body posture can include information such as forward head posture, pelvic tilt, lumbar lordosis, sacral slope, etc. In some implementations, the estimated body posture includes information describing whether the user is in a forward head posture based on a predetermined threshold of forward head position. For example, the estimated body posture can include information affirming that the user is in a forward head posture if a calculated craniovertebral angle is below a predetermined value.

At step 608, the method 600 includes outputting the estimated body posture to a user interface. In some implementations, the estimated body posture is outputted to a graphical user interface on a display of a head-mounted display. The estimated body posture can be presented to inform the user whether they are in a poor body posture. In further implementations, the method 600 includes outputting recommendations describing corrective posture actions that the user can take to prevent and/or correct a poor body posture. The recommendation can be in the form of instructional text, images, and/or videos. In some implementations, the estimated body posture is outputted to the user interface if a predetermined criteria is met. For example, the estimated body posture can be outputted when the system detects that the user is in an upright position. Various sensors can be implemented to detect whether the user is in a reclining position. For example, cameras can be used to detect a reclining position based on captured image data. IMUs can also be used to detect movements that are associated with a reclining activity.

Additionally or alternatively, the artificial neural network can be trained during the body posture estimation process to tailor the process to a given user. The artificial neural network can be trained via a supervised learning process with target body posture outputs provided by the user. For example, the artificial neural network can be used to predict a body posture given sensor data received from sensors during operation of a head-mounted display by a user. At step 610, the method 600 optionally includes receiving input from the user indicating accuracy of the estimated body posture. The input can be received through various input devices. In some implementations, the input is received through the tracking of hand gestures by the user via a hand-tracking camera. In some implementations, the input is received from eye-tracking cameras used for gaze detection. At step 612, the method 600 optionally includes computing a loss value using a loss function based on the estimated body posture and the received input from the user. The received input serves as the target output that can be used with the predicted body posture to compute a loss value based on a loss function. The computed loss value describes a difference between the predicted body posture and the provided target output. Different types of loss functions, including L1 and L2 loss functions, can be utilized. At step 614, the method 600 includes adjusting the machine learning model based on the computed loss value. For example, the computed loss value can be used to adjust the parameters and/or weights of an artificial neural network.

The examples disclosed herein provide for body posture sensing that can be employed with little to no inconvenience to a user. In comparison to current posture sensing devices, which include uncomfortable wearables under the user's clothing, head-mounted devices can be designed as everyday wearables comparable to regular eyeglasses. Head-mounted devices with body posture estimation functionalities can provide information and cues to the user regarding his or her posture using different types of sensors, some or all of which can be implemented in a head-mounted device for other applications. The multi-modal data signals from the different types of sensors can be applied to a machine learning model that works to aggregate and discover correlations among the data signals to provide accurate body posture estimations.

In some embodiments, the methods and processes described herein may be tied to a computing system of one or more computing devices. In particular, such methods and processes may be implemented as a computer-application program or service, an application-programming interface (API), a library, and/or other computer-program product.

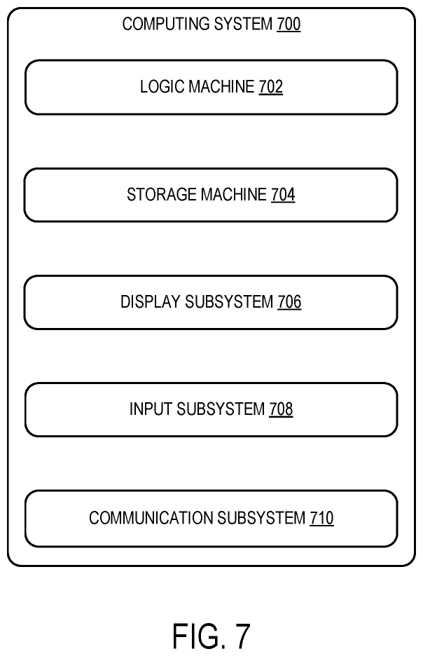

FIG. 7 schematically shows a non-limiting embodiment of a computing system 700 that can enact one or more of the methods and processes described above. For example, the computing system 700 may be implemented onboard a head-mounted device as a controller for executing instructions to perform head posture estimation. Computing system 700 is shown in simplified form. Computing system 700 may take the form of one or more personal computers, server computers, tablet computers, home-entertainment computers, network computing devices, gaming devices, mobile computing devices, mobile communication devices (e.g., smart phone), and/or other computing devices.

Computing system 700 includes a logic machine 702 and a storage machine 704. Computing system 700 may optionally include a display subsystem 706, input subsystem 708, communication subsystem 710, and/or other components not shown in FIG. 7.

Logic machine 702 includes one or more physical devices configured to execute instructions. For example, the logic machine 702 may be configured to execute instructions that are part of one or more applications, services, programs, routines, libraries, objects, components, data structures, or other logical constructs. Such instructions may be implemented to perform a task, implement a data type, transform the state of one or more components, achieve a technical effect, or otherwise arrive at a desired result.

The logic machine 702 may include one or more processors configured to execute software instructions. Additionally or alternatively, the logic machine 702 may include one or more hardware or firmware logic machines configured to execute hardware or firmware instructions. Processors of the logic machine 702 may be single-core or multi-core, and the instructions executed thereon may be configured for sequential, parallel, and/or distributed processing. Individual components of the logic machine optionally may be distributed among two or more separate devices, which may be remotely located and/or configured for coordinated processing. Aspects of the logic machine 702 may be virtualized and executed by remotely accessible, networked computing devices configured in a cloud-computing configuration.

Storage machine 704 includes one or more physical devices configured to hold instructions executable by the logic machine to implement the methods and processes described herein. When such methods and processes are implemented, the state of storage machine 704 may be transformed—e.g., to hold different data.

Storage machine 704 may include removable and/or built-in devices. Storage machine 704 may include optical memory (e.g., CD, DVD, HD-DVD, Blu-Ray Disc, etc.), semiconductor memory (e.g., RAM, EPROM, EEPROM, etc.), and/or magnetic memory (e.g., hard-disk drive, floppy-disk drive, tape drive, MRAM, etc.), among others. Storage machine 704 may include volatile, nonvolatile, dynamic, static, read/write, read-only, random-access, sequential-access, location-addressable, file-addressable, and/or content-addressable devices.

It will be appreciated that storage machine 704 includes one or more physical devices. However, aspects of the instructions described herein alternatively may be propagated by a communication medium (e.g., an electromagnetic signal, an optical signal, etc.) that is not held by a physical device for a finite duration.

Aspects of logic machine 702 and storage machine 704 may be integrated together into one or more hardware-logic components. Such hardware-logic components may include field-programmable gate arrays (FPGAs), program- and application-specific integrated circuits (PASIC/ASICs), program- and application-specific standard products (PSSP/ASSPs), system-on-a-chip (SOC), and complex programmable logic devices (CPLDs), for example.

The terms “module,” “program,” and “engine” may be used to describe an aspect of computing system 700 implemented to perform a particular function. In some cases, a module, program, or engine may be instantiated via logic machine 702 executing instructions held by storage machine 704. It will be understood that different modules, programs, and/or engines may be instantiated from the same application, service, code block, object, library, routine, API, function, etc. Likewise, the same module, program, and/or engine may be instantiated by different applications, services, code blocks, objects, routines, APIs, functions, etc. The terms “module,” “program,” and “engine” may encompass individual or groups of executable files, data files, libraries, drivers, scripts, database records, etc.

It will be appreciated that a “service”, as used herein, is an application program executable across multiple user sessions. A service may be available to one or more system components, programs, and/or other services. In some implementations, a service may run on one or more server-computing devices.

When included, display subsystem 706 may be used to present a visual representation of data held by storage machine 704. This visual representation may take the form of a graphical user interface (GUI). As the herein described methods and processes change the data held by the storage machine, and thus transform the state of the storage machine, the state of display subsystem 706 may likewise be transformed to visually represent changes in the underlying data. Display subsystem 706 may include one or more display devices utilizing virtually any type of technology. Such display devices may be combined with logic machine 702 and/or storage machine 704 in a shared enclosure, or such display devices may be peripheral display devices.

When included, input subsystem 708 may comprise or interface with one or more user-input devices such as a keyboard, mouse, touch screen, or game controller. In some embodiments, the input subsystem may comprise or interface with selected natural user input (NUI) componentry. Such componentry may be integrated or peripheral, and the transduction and/or processing of input actions may be handled on- or off-board. Example NUI componentry may include a microphone for speech and/or voice recognition; an infrared, color, stereoscopic, and/or depth camera for machine vision and/or gesture recognition; a head tracker, eye tracker, accelerometer, and/or gyroscope for motion detection and/or intent recognition; as well as electric-field sensing componentry for assessing brain activity.

When included, communication subsystem 710 may be configured to communicatively couple computing system 700 with one or more other computing devices. Communication subsystem 710 may include wired and/or wireless communication devices compatible with one or more different communication protocols. As non-limiting examples, the communication subsystem 710 may be configured for communication via a wireless telephone network, or a wired or wireless local- or wide-area network. In some embodiments, the communication subsystem 710 may allow computing system 700 to send and/or receive messages to and/or from other devices via a network such as the Internet.

Another aspect includes a head-mounted device for postural alignment correction, the head-mounted device comprising a display, a plurality of sensors comprising an inertial measurement unit, and a controller. The controller comprises instructions executable to control the head-mounted device to receive inertial measurement data from the inertial measurement unit, input the received inertial measurements into a machine learning model, and receive an estimated body posture from the machine learning model. In this aspect, additionally or alternatively, the body posture comprises a head posture, and the machine learning model is an artificial neural network that has been trained to estimate the head posture by calculating a craniovertebral angle. In this aspect, additionally or alternatively, the plurality of sensors further comprises one or more cameras, and the artificial neural network has been trained to estimate the body posture using image data from the one or more cameras. In this aspect, additionally or alternatively, the neural network has been trained based on an average human population, and estimating the body posture comprises computing a loss value using a loss function and adjusting the trained neural network based on the computed loss value. In this aspect, additionally or alternatively, computing the loss value using the loss function comprises a supervised learning process that includes receiving an input from a user indicating accuracy of the estimated body posture. In this aspect, additionally or alternatively, the controller further comprises instructions executable to output information to a user using the display, wherein the information indicates the estimated body posture. In this aspect, additionally or alternatively, the presented information includes recommendations on corrective posture actions.

Another aspect includes a head-mounted device for postural alignment correction, the head-mounted device comprising a display, a plurality of sensors comprising a camera, and a controller. The controller comprises instructions executable to control the head-mounted device to receive image data from the camera, input the received image data into an artificial neural network, and receive an estimated body posture from the artificial neural network. In this aspect, additionally or alternatively, the plurality of sensors comprises one or more of a downward-facing camera and a forward-facing camera, and wherein the image data comprises stereoscopic image data. In this aspect, additionally or alternatively, the estimated body posture comprises an estimated head posture, the plurality of sensors further comprises an inertial measurement unit, and the neural network has been trained to estimate the body posture by calculating a craniovertebral angle using inertial measurement data from the inertial measurement unit and image data from the camera. In this aspect, additionally or alternatively, the artificial neural network has been trained to estimate the body posture using the image data based on a portion of a body of a user that is in view of the camera. In this aspect, additionally or alternatively, the artificial neural network has been trained to estimate a reclining position of a user using the image data from the camera. In this aspect, additionally or alternatively, the artificial neural network has been trained based on an average human population, and estimating the body posture comprises computing a loss value using a loss function and adjusting the trained neural network based on the calculated loss. In this aspect, additionally or alternatively, computing the loss value using the loss function comprises a supervised learning process that includes receiving an input from a user indicating accuracy of the estimated body posture. In this aspect, additionally or alternatively, the controller further comprises instructions executable to present information to a user using the display, wherein the information indicates the estimated body posture. In this aspect, additionally or alternatively, the presented information includes recommendations on corrective posture actions.

Another aspect includes, on a head-mounted computing device, a method for postural alignment correction, the method comprising receiving inertial measurement data from an inertial measurement unit, inputting the received inertial measurements into an artificial neural network, receiving an estimated body posture from the artificial neural network, outputting the estimated body posture to a user interface, receiving input from a user indicating accuracy of the estimated body posture, computing a loss value using a loss function based on the estimated body posture and the received input from the user, and adjusting the artificial neural network based on the computed loss value. In this aspect, additionally or alternatively, the estimated body posture comprises an estimated head posture, and wherein the artificial neural network has been trained to estimate the head posture by calculating a craniovertebral angle. In this aspect, additionally or alternatively, the method further comprises receiving image data from a camera comprising one or more of a downward-facing camera and a forward-facing camera and inputting the image data into the artificial neural network. In this aspect, additionally or alternatively, the method further comprises outputting recommendations on corrective posture actions to the user interface.

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. A head-mounted device for postural alignment correction, the head-mounted device comprising:
a display;
a plurality of sensors comprising an inertial measurement unit; and
a controller comprising instructions executable to control the head-mounted device to:
receive inertial measurement data from the inertial measurement unit;
input the received inertial measurements into a machine learning model; and
receive an estimated body posture from the machine learning model.

2. The head-mounted device of claim 1, wherein the body posture comprises a head posture, and wherein the machine learning model is an artificial neural network that has been trained to estimate the head posture by calculating a craniovertebral angle.

3. The head-mounted display of claim 1, wherein the plurality of sensors further comprises one or more cameras, and wherein the artificial neural network has been trained to estimate the body posture using image data from the one or more cameras.

4. The head-mounted device of claim 1, wherein the neural network has been trained based on an average human population, and wherein estimating the body posture comprises:
computing a loss value using a loss function; and
adjusting the trained neural network based on the computed loss value.

5. The head-mounted device of claim 4, wherein computing the loss value using the loss function comprises a supervised learning process that includes receiving an input from a user indicating accuracy of the estimated body posture.

6. The head-mounted device of claim 1, wherein the controller further comprises instructions executable to output information to a user using the display, wherein the information indicates the estimated body posture.

7. The head-mounted device of claim 1, wherein the presented information includes recommendations on corrective posture actions.

8. A head-mounted device for postural alignment correction, the head-mounted device comprising:
a display;
a plurality of sensors comprising a camera; and
a controller comprising instructions executable to control the head-mounted device to:
receive image data from the camera;
input the received image data into an artificial neural network; and
receive an estimated body posture from the artificial neural network.

9. The head-mounted device of claim 8, wherein the plurality of sensors comprises one or more of a downward-facing camera and a forward-facing camera, and wherein the image data comprises stereoscopic image data.

10. The head-mounted device of claim 8, wherein:
the estimated body posture comprises an estimated head posture;
the plurality of sensors further comprises an inertial measurement unit, and
the neural network has been trained to estimate the body posture by calculating a craniovertebral angle using inertial measurement data from the inertial measurement unit and image data from the camera.

11. The head-mounted device of claim 8, wherein the artificial neural network has been trained to estimate the body posture using the image data based on a portion of a body of a user that is in view of the camera.

12. The head-mounted device of claim 8, wherein the artificial neural network has been trained to estimate a reclining position of a user using the image data from the camera.

13. The head-mounted device of claim 8, wherein the artificial neural network has been trained based on an average human population, and wherein estimating the body posture comprises:
computing a loss value using a loss function; and
adjusting the trained neural network based on the calculated loss.

14. The head-mounted device of claim 13, wherein computing the loss value using the loss function comprises a supervised learning process that includes receiving an input from a user indicating accuracy of the estimated body posture.

15. The head-mounted device of claim 8, wherein the controller further comprises instructions executable to present information to a user using the display, wherein the information indicates the estimated body posture.

16. The head-mounted device of claim 8, wherein the presented information includes recommendations on corrective posture actions.

17. On a head-mounted computing device, a method for postural alignment correction, the method comprising:
receiving inertial measurement data from an inertial measurement unit;
inputting the received inertial measurements into an artificial neural network;
receiving an estimated body posture from the artificial neural network;
outputting the estimated body posture to a user interface;
receiving input from a user indicating accuracy of the estimated body posture;
computing a loss value using a loss function based on the estimated body posture and the received input from the user; and
adjusting the artificial neural network based on the computed loss value.

18. The method of claim 17, wherein the estimated body posture comprises an estimated head posture, and wherein the artificial neural network has been trained to estimate the head posture by calculating a craniovertebral angle.

19. The method of claim 17, further comprising:
receiving image data from a camera comprising one or more of a downward-facing camera and a forward-facing camera; and
inputting the image data into the artificial neural network.

20. The method of claim 17, further comprising:
outputting recommendations on corrective posture actions to the user interface.
